(19) United States
(12) Patent Application Publication
(10) Pub. No.: US 2016/0257917 A1
(43) Pub. Date: Sep. 8, 2016

(54) AC Brew

(71) Applicant: Roy Griffith, Crystal City, TX (US)

(72) Inventor: Roy Griffith, Crystal City, TX (US)

(21) Appl. No.: 14/639,115

(22) Filed: Mar. 4, 2015

(51) Int. Cl. C12J 1/08; A61K 36/73; A23L 2/02; A23L 2/60; A23L 2/56

(52) U.S. Cl. CPC C12J 1/08; A23L 2/60; A23L 2/56; A23L 2/02; A61K 36/73; A23V 2002/00

(57) ABSTRACT

The current invention disclosure pertains to a health beverage comprise of apple cider vinegar, stevia extracts and tea unlike the existing apple cider vinegar beverages in market the current invention beverage has a unique combination that has eliminated the sour taste of apple cider vinegar without destroying its health benefits.

### CROSS REFERENCE TO RELATED APPLICATIONS

Not applicable.

### FEDERALLY SPONSORED RESEARCH AND DEVELOPMENT

Not applicable.

### MICROFICHE APPENDIX

Not applicable.

### BACKGROUND OF THE INVENTION

1. Field of the Invention

The invention belongs to the technical field of beverages. The invented drink is a health beverage that is made up of a specific composition of tea, apple cider vinegar, and stevia.

2. Background of Invention

With the improvement of people's living standards and material possessions, the demand increases for the development of the beverage industry's ever-changing, colorful varieties of beverages especially those with health functions. A drink that has become a new favorite beverage is a tea-vinegar drink.

Apple cider vinegar is a renowned health drink. Apple cider vinegar, also known as cider vinegar or ACV, is a type of vinegar made from cider or apple must and has a pale to medium amber color. A mixture of apple cider vinegar and water is an effective remedy for diarrhea. Apple cider vinegar contains pectin, which is highly effective in soothing intestinal spasms. Apple cider vinegar also cures hiccups and sore throat, as the acidic environment created by apple cider vinegar kills the germs. It can also cure high cholesterol, diabetes, acne, and sores.

In 1958, Dr. Jarvis wrote a book about the health benefits that can be obtained by use of apple cider vinegar. According to his recommendations, users should mix apple cider vinegar with honey and take a tablespoon a day. Since the 1970s, apple cider vinegar had risen in popularity. After reading his book, proponents created a weight loss plan in which the key ingredient is apple cider vinegar.

Furthermore, many health practitioners believe that apple cider helps to balance out the pH balance in the body. Blood is normally more alkaline, if individuals are suffering from an illness or overwork, the pH balance can be thrown off. By taking apple cider vinegar, proponents believe that the body's natural balance will be restored.

Despite the widely known health benefits of apple cider vinegar, this drink is extremely sour, making it difficult for individuals to consume.

In order to overcome this drawback, and to increase the consumption of apple cider vinegar by individuals, there is a need to improve the taste of apple cider vinegar.

Existing prior arts do disclose many beverages having apple cider vinegar as main ingredient such as U.S. Pat. No. 6,932,988 B2, CN 103504295 A and CN 102835711 B, but none of the prior art focusses on improving the taste of apple cider vinegar without impacting its inherent qualities.

The current invention discloses combination of specific ingredients that are mixed with apple cider vinegar to make it a tasteful and healthy beverage.

The current invention has improved the taste of apple cider vinegar by adding tea and stevia to it. This combination has not only improved the taste, but the combination is rich in catechins. Catechins help in the elimination of free radicals in the human body, is a natural pigment protective agent, can inhibit cholesterol, and soften blood vessels. Polyphenols have anti-aging, antioxidant, antimicrobial, and disease prevention roles. This Tea Vinegar combination also has a refreshing taste, supports diuretic detoxification, and eliminates fatigue.

However, vinegar drink tea varieties on the market are rare; the current invention is not only a perfect combination of health beverage ingredients, but is exceptionally good in taste.

### BRIEF SUMMARY OF THE INVENTION

The following summary is provided to facilitate an understanding of some of the innovative features unique to the disclosed embodiment and is not intended to be a full description. A full appreciation of the various aspects of the embodiments disclosed herein can be gained by taking the entire specification, claims and abstract as a whole.

The current invention disclosure discloses health beverage having a unique combination of apple eider vinegar, tea and stevia.

The objective of the current invention is to provide a tasty and healthy beverage.

It is also an objective of the invention to provide a medicinal beverage that assists in curing sore throats, bacterial infections, hiccups, intestinal spasms, high cholesterol, diabetes, acne, and sores.

It is also an objective of the invention to provide a beverage that is easy to make.

It is also an objective of the invention to provide a beverage that is suitable for members of all age groups.

It is also an objective of the invention to provide a beverage that improves the taste of apple cider vinegar without destroying its properties.

It is also an objective of the invention to provide an apple cider vinegar beverage that can be stored or refrigerated.

It is further an objective of the invention to provide a health beverage that can be easily incorporated in the daily routine of individuals.

Other objectives of the invention will become apparent to those skilled in the art once the invention has been shown and described. These objectives are not to be construed as limitations of the applicant's invention, but are merely aimed to suggest some of the many benefits that may be realized by the apparatus of the present application, and with its many embodiments. In addition to the various objects and advantages of the present invention described with some degree of specificity above, it should be obvious that additional objects and advantages of the present invention will become more readily apparent to those persons who are skilled in the relevant art from the following more detailed description of the invention.

### DETAILED DESCRIPTION OF THE INVENTION

The embodiments of the present disclosure described below are not intended to be exhaustive or to limit the disclosure to the precise forms disclosed in the following detailed description. Rather, the embodiments are chosen and described so that others skilled in the art may appreciate and understand the principles and practices of the present disclosure.

The following embodiments and examples (if any) which are incorporated into, form part of this disclosure, embodiments of the invention together with the description, serve to explain the principles of the invention.

The current invention provides a health beverage that can be easily canned and sold in market.

Today a large variety of health drinks having apple cider vinegar are available in market but they are bitter in taste. The purpose of the present invention is to provide a tasty and easy to consume health drink having apple cider vinegar.

The health beverage of the current invention suppresses the bitter taste of apple cider vinegar without impacting the quality and properties of apple cider vinegar.

The health benefits of apple cider vinegar in itself are huge and it has been used for centuries to cure a variety of illnesses and also used to delay the aging process. Apple cider vinegar helps protect body cells from free radicals damage. It also contains vitamins E, A, C, beta carotene, 20 different minerals and trace elements. Pectin in apple cider vinegar helps in reducing the harmful LDL cholesterol. Therefore, the good quality of the organic cider vinegar is very important. The active ingredient caffeine can, boost brain power and even possibly prevent neurological disorders such as Parkinson's or Alzheimer's. For people who do not like or tolerate caffeine can be offered with Green Tea, alternatively, apple cider vinegar lemonade.

Green tea is considered in Asian countries as a remedy, it has numerous health benefits and healing substances, it contains a natural antibiotic. The suggested intake of apple cider vinegar and green tea is preferably in the morning to or after breakfast. Due to its health-promoting properties, this combination can be used as a beverage to avoid premature aging.

The stevia extracts used in current invention also has special significance as raw leaves of the stevia plant are approximately 40 times sweeter than sugar, and the powdered sweetener derived from them is up to 200-300 times sweeter. What this means, is that just a tiny bit of stevia will suffice for making sugar free recipes.

Stevia is available in various forms in market such as white extract powder, dried stevia leaves, stevia liquid extract, or as small pellets. Any form can be used for making the health beverage disclosed herein.

Stevia extracts are highly beneficial for people suffering from diabetes and obesity. Stevia extracts are devoid of any calories and sucrose.

The invention provides a Health beverage composition essentially having apple cider vinegar, stevia and tea. Hence it is highly beneficial for health of any individual.

Preferred composition of the health beverage is Water, Cold Brew Tea Bags, Bragg's Organic Apple Cider Vinegar, Kal Pure Stevia Extract.

This composition may be substituted by use of wide variety of teas to inculcate the desired properties in the beverage.

Another aspect of the invention involves process for the preparation of health beverage which includes following steps:

The process of making health beverage comprises the steps of:

1. Refrigeration of 1 (one) gallon of water until cool.

2. Addition of 2 (two) cold brew tea bags and brewed for 15 min. Steeping to be done every three minutes.

3. Pouring 23 fl oz. of the water into serving container.

4. Adding 1½ Tablespoons of Bragg's Organic ACV and stirring the mixture.

5. Adding 80 milligrams of Kat Pure Stevia Extract and stirring the mixture until fully dissolved.

Although specific embodiments have been illustrated and described herein, it will be appreciated by those of ordinary skill in the art that any arrangement, which is calculated to achieve the same purpose, may be substituted for the specific embodiment shown. This application is intended to cover any adaptations or variations of the present invention.

Although the invention has been explained in relation to its preferred embodiment, it is to be understood that many other possible modifications and variations can be made without departing from the spirit and scope of the invention.

I claim:

1. A health beverage comprising:
Bragg's organic apple cider vinegar;
Kal pure stevia extract;
Cold brew tea bags; and
Water.

2. The health beverage, as claimed in claim 1, is produced by the following method:
Step 1: Refrigeration, wherein 1 (one) gallon of water is refrigerated.
Step 2: Steeping, wherein 2 (two) cold brew tea bags are steeped and brewed for 15 min.
Step 3: Pouring, wherein 23 fl oz. of the water is poured into the serving container.
Step 4: Mixing, wherein 1½ Tablespoons of Bragg's Organic ACV to water is added, along with 80 milligrams of Kal Pure Stevia extract, the mixture is stirred until fully dissolved.
